(19) United States
(12) Patent Application Publication
(10) Pub. No.: US 2007/0074718 A1
(43) Pub. Date: Apr. 5, 2007

(54) Metered dose inhaler having spacing device

(75) Inventor: Gary R. Austin, Euclid, OH (US)

Correspondence Address: PEARNE & GORDON LLP, 1801 EAST 9TH STREET, SUITE 1200, CLEVELAND, OH, 44114-3108, US

(73) Assignee: SP Medical LLC

(21) Appl. No.: 11/542,835

(22) Filed: Oct. 4, 2006

Related U.S. Application Data
(60) Provisional application No. 60/723,500, filed on Oct. 4, 2005.

(51) Int. Cl. A61M 11/00

(52) U.S. Cl. 128/200.23

(57) ABSTRACT

A medication inhaler is provided for delivery of a medication to a patient from a canister having a valve stem, such as a MDI canister. The medication inhaler includes a chamber body having a fresh air inlet and a hollow spacer chamber. The inhaler further includes a canister retainer configured to move the canister between operational and stored positions, and an actuation lever for actuation of the valve stem to deliver a medication fluid into the hollow spacer chamber. In one example, the hollow spacer chamber tapers. In addition or alternatively, the chamber body includes a nozzle having an outlet, and a fresh air inlet having an outlet proximate the outlet of the nozzle. In addition or alternatively, the medication inhaler includes a canister adapter for accommodation of various size canisters. In addition or alternatively, the chamber body includes an inlet portion having a geometry for guiding the valve stem.

### RELATED APPLICATIONS

This application claims the benefit of U.S. Provisional Application No. 60/723,500, filed on Oct. 4, 2005, the entire disclosure of which is hereby incorporated herein by reference.

### FIELD OF THE INVENTION

The present invention relates to a pressurized metered dose inhaler for effectively delivering medications by inhalation through the mouth of patients having a spacing device for more effectively mixing the medication and air, and an easy to operate lever mechanism.

### BACKGROUND OF THE INVENTION

Inhalers are commonly used to dispense an aerosol for treatment, or alleviation of the effects of respiratory complaints, such as asthma. One of the most convenient choices of treatment of respiratory complaints has been the inhalation of a medicament from a drug solution or suspension in a metered dosed inhaler (MDI), such as a pressurized metered dose inhaler (pMDI).

Standard metered dosage inhalers have effectively produced an aerosol of a medicament in a predetermined dosage for delivery to the lungs. However, awkward dispensing mechanisms and inefficient delivery systems decrease the likelihood that the medicament effectively reaches the patients lungs. Known inhalation devices typically include a tubular housing or sleeve in which a pressurized aerosol container is located and a mouthpiece or nozzle leading out of the tubular housing. In use, the aerosol container is placed into the housing, which is then held by the patient in a more or less upright position, and the mouthpiece or nozzle of the inhalation device is placed in the mouth or nostril of the patient. The patient inhales through either the mouthpiece or nozzle while simultaneously releasing the medicament from the aerosol container. With such devices, the patient releases the medicament either manually by pressing on a bottom surface of the aerosol container, or using an actuator that exerts a force on the bottom surface of the aerosol container.

Even if a patient times inhalation with the dispersal of the medicament from the pMDI, the amount of medicament reaching the lungs is inconsistent, depending on factors such as how much of the medicament is dispersed and entrained in the stream of air entering the patient's lungs and how much of the medicament is deposited on surfaces of the inhalation apparatus and on the mouth and oropharyngeal area of the patient. Furthermore, deposits in the mouth and oropharyngeal area of the patient can cause complications, such as candidiasis, and leave an unpleasant aftertaste. In addition, many patients using inhalation devices have practical problems with the use of typical inhalers, including difficulty with compressing the metered dose inhaler canister, problems timing inhalation with the dispersal of medicament, and inability to inhale the complete dose of medicament in a single breath, particularly young, elderly, or patients who suffer from asthma and dysphonia or thrush from inhaled corticosteroids. In practice, it is common to see patients activating their metered dose inhalers multiple times, although the metered dose should have been dispensed in a single activation. This is a typical response of patients to the difficulty and inconsistency of medicament delivery, undermining the effectiveness of the pMDI, which can lead to over-medication, under-medication, or waste and higher than necessary costs for treatment.

The typical solution to the problem of inconsistent medicament delivery has been to provide a “spacer” or “chamber” within the inhalation device. A spacer or chamber is merely a reservoir for air. The metered dose of medicament, usually an atomized mist or vapor, mixes with the reservoir of air before being inhaled by the patient, reducing the inconsistency of delivery due to inhalation and actuation timing difficulties. An additional improvement incorporated into many of the devices with spacers has been a simple valve mechanism designed to hold the medicament in a contained space and to allow the patient to inhale the medicament in more than one breath.

A further problem is the ease of compression of pMDI canisters. One spacer improves the ease of compression by allowing the patient or another to use both thumbs on a pull handle and the forefingers on the pMDI canister. Another solution is to provide a lever that extends along the length of the chamber, for example, as shown and described in U.S. Pat. Nos. 6,523,536 and 6,698,422. Such devices tend to be bulky and difficult to carry. Generally, the pMDI canister must be removed from the spacer after each use and carried separately. Moreover, inserting the canister into the spacer can be difficult for many patients, especially elderly persons, since the valve stem must be inserted into a very small hole. Failure to properly insert the canister into the spacer can result in ineffective actuation of the pMDI or damage to the stem, rendering the canister useless.

### BRIEF SUMMARY OF THE INVENTION

The following presents a simplified summary of the invention in order to provide a basic understanding of some aspects of the invention. This summary is not an extensive overview of the invention. It is intended to identify neither key nor critical elements of the invention nor delineate the scope of the invention. Its sole purpose is to present some concepts of the invention in a simplified form as a prelude to the more detailed description that is presented later.

In accordance with one aspect, a medication inhaler is provided for delivery of a medication from a MDI canister to a patient where the MDI canister has a valve stem. The medication inhaler includes a chamber body having a proximal end, a distal end, and a fresh air inlet. The chamber body also includes a hollow spacer chamber located within the chamber body and tapering from a relatively larger cross-sectional area adjacent the proximal end to a relatively smaller cross-sectional area adjacent the distal end, and an inlet portion for receiving the valve stem of the MDI canister. The inhaler further includes a canister retainer configured to retain the MDI canister in an operational position relative to the chamber body, and an actuation lever pivotally attached to the chamber body. The actuation lever is configured to actuate the valve stem of the MDI canister to deliver a medication fluid into the hollow spacer chamber. The inhaler further includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece. The fresh air inlet provides fluid communication between the atmosphere and the hollow spacer chamber to deliver a volume of fresh air from the atmosphere into the hollow spacer chamber upon inhalation by the patient for mixture with the medication fluid such that the medication fluid is generally uniformly distributed within the volume of fresh air before the medication fluid passes from the hollow spacer chamber and into the mouthpiece.

In accordance with another aspect, a medication inhaler is provided for delivery of a medication from a MDI canister to a patient where the MDI canister has a valve stem. The medication inhaler includes a chamber body having a proximal end, a distal end, a hollow spacer chamber located within the chamber body, and an inlet portion for receiving the valve stem of the MDI canister. The chamber body also includes a nozzle in fluid communication with the inlet portion and the hollow spacer chamber, the nozzle having an outlet, and a fresh air inlet in fluid communication with the atmosphere and the hollow spacer chamber, the fresh air inlet having an outlet proximate the outlet of the nozzle. The medication inhaler includes a canister retainer configured to retain the MDI canister in an operational position relative to the chamber body, and an actuation lever pivotally attached to the chamber body. The actuation lever is configured to actuate the valve stem of the MDI canister to deliver a medication fluid into the hollow spacer chamber. The medication inhaler includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece.

In accordance with another aspect, a medication inhaler is provided for delivery of a medication from a MDI canister to a patient from at least one of a first MDI canister having a first size and a second MDI canister having a second size, each of the first and second MDI canisters having a valve stem. The medication inhaler includes a chamber body having a proximal end, a distal end, a hollow spacer chamber located within the chamber body, and an inlet portion for receiving the valve stem of one of the MDI canisters. The medication inhaler further includes a canister retainer configured to retain either of the MDI canisters in an operational position relative to the chamber body, and an actuation lever pivotally attached to the chamber body. The actuation lever is configured to actuate the valve stem of the MDI canister retained by the canister retainer to deliver a volume of medication fluid into the hollow spacer chamber. The medication inhaler further includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece. The medication inhaler further includes a canister adapter attached to the canister retainer and adjustable from a first configuration to a second configuration. The first configuration is adapted to position the valve stem of the first MDI canister adjacent the inlet portion, and the second configuration is adapted position the valve stem of the second MDI canister adjacent the inlet portion.

In accordance with yet another aspect, a medication inhaler is provided for delivery of a medication from a MDI canister to a patient where the MDI canister has a valve stem. The medication inhaler includes a chamber body having a proximal end, a distal end, and a hollow spacer chamber located within the chamber body. The chamber body further includes an inlet portion having a first end for receiving the valve stem of the MDI canister and a second end for engagement with the valve stem. The inlet portion tapers from a relatively larger cross-sectional area adjacent the first end to a relatively smaller cross-sectional area adjacent the second end. The medication inhaler further includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece. The medication inhaler further includes a canister retainer configured to retain the MDI canister in an operational position relative to the chamber body, and an actuation lever pivotally attached to the chamber body. The actuation lever is configured to move the valve stem of the MDI canister into contact with the second end of the inlet portion to deliver a medication fluid into the hollow spacer chamber.

In accordance with yet another aspect, a medication inhaler is provided for delivery of a medication from a MDI canister to a patient where the MDI canister has a valve stem. The medication inhaler includes a chamber body having a proximal end, a distal end, a hollow spacer chamber located within the chamber body, and an inlet portion having a first end for receiving the valve stem of the MDI canister and a second end for engagement with the valve stem. The medication inhaler further includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece. The medication inhaler further includes a canister retainer configured to retain the MDI canister in an operational position relative to the chamber body and an actuation lever pivotally attached to the chamber body. The actuation lever is configured to move the valve stem into contact with the inlet portion. A geometry of the inlet portion is configured to guide the valve stem towards the second end thereof, and engagement of the valve stem with the second end causes actuation of the valve stem to deliver a medication fluid into the hollow spacer chamber.

In accordance with still yet another aspect, a medication inhaler is provided for delivery of a medication from a MDI canister to a patient, where the MDI canister has an upper end, a lower end, and a valve stem disposed at the lower end. The medication inhaler includes a chamber body having a proximal end, a distal end, a hollow spacer chamber located within the chamber body, and an inlet portion for receiving the valve stem of the MDI canister. The medication inhaler further includes a mouthpiece shaped to fit a mouth of a patient and connected to the proximal end of the chamber body, and a one-way valve disposed between the mouthpiece and the hollow spacer chamber and configured to provide one-way fluid communication between the hollow spacer chamber and the mouthpiece. The medication inhaler further includes an actuation lever having an elongated opening and being pivotally attached to the chamber body. The actuation lever is configured to actuate the valve stem of the MDI canister retained by the canister retainer to deliver a volume of medication fluid into the hollow spacer chamber. The medication inhaler further includes a canister retainer configured to retain the MDI canister. The canister retainer is pivotally attached to the actuation lever and is movable between a stored position and an operational position. The stored position is a lowered position such that at least a portion of the upper end of the MDI canister is received within the elongated opening of the actuation lever. The operational position is a raised position such that the valve stem of the MDI canister is adjacent the inlet portion of the chamber body.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 illustrates a perspective view of an example medication inhaler in accordance with an aspect of the present invention;

FIG. 2 is similar to FIG. 1, but shows an exploded view;

FIG. 3A illustrates a front view of the example medication inhaler of FIG. 1;

FIG. 3B is similar to FIG. 3A, but shows a top view;

FIG. 3C is similar to FIG. 3A, but shows a left side view;

FIG. 3D is similar to FIG. 3A, but shows a right side view;

FIG. 4 illustrates a sectional view along line 4-4 of FIG. 3B;

FIG. 5A illustrates an example canister adapter attached to an example canister retainer in accordance with another aspect of the present invention;

FIG. 5B is similar to FIG. 5A, but shows the canister adapted in another configuration;

FIG. 6 is similar to FIG. 3A, but shows the medication inhaler configured for use with an alternate MDI canister;

FIG. 7 is similar to FIG. 2, but shows an example alternate canister adapter for use with an example alternate canister retainer;

FIG. 8 is similar to FIG. 3C, but shows the chamber body after removal of the mouthpiece in the direction of line 8-8 of FIG. 4;

FIG. 9 is similar to FIG. 4, but illustrates an example interaction between fresh air and medication within the chamber body;

FIG. 10A is similar to FIG. 3A, but shows the medical inhaler in a stored configuration;

FIG. 10B is similar to FIG. 10A, but shows a top view;

FIG. 10C is similar to FIG. 10A, but shows a left side view;

FIG. 10D is similar to FIG. 10A, but shows a right side view; and

FIG. 11 illustrates a sectional view along line 11-11 of FIG. 10B.

### DESCRIPTION OF EXAMPLE EMBODIMENTS

An example embodiment of a device that incorporates aspects of the present invention is shown in the drawings. It is to be appreciated that the shown example is not intended to be a limitation on the present invention. For example, one or more aspects of the present invention can be utilized in other embodiments and even other types of devices.

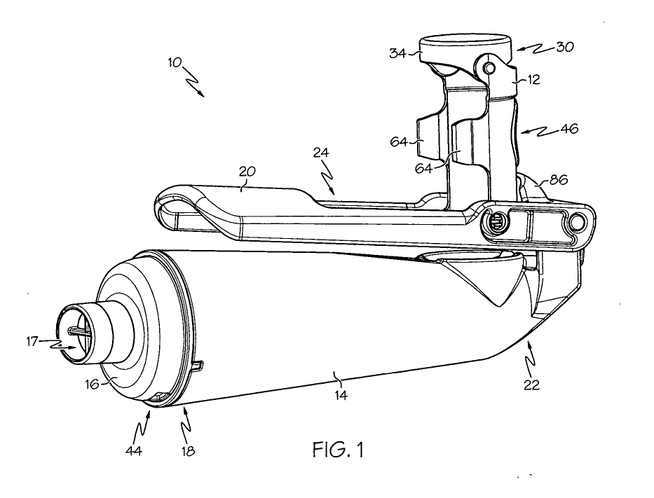

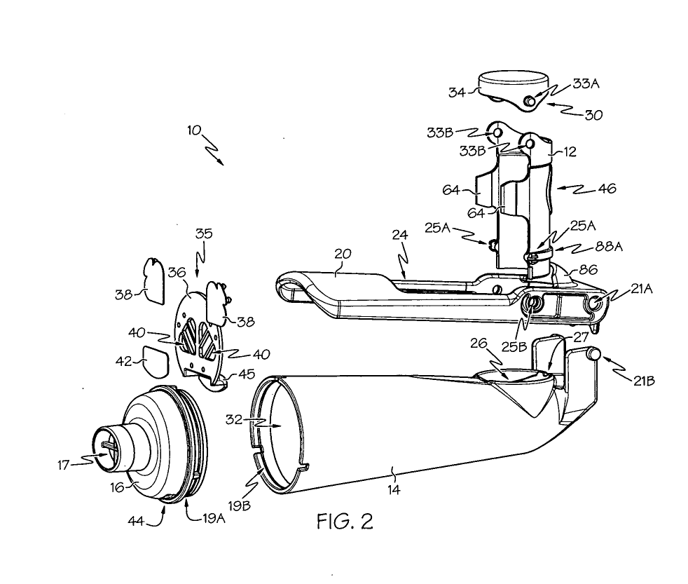

Turning to the shown example of FIGS. 1 and 2, the present invention provides a medication inhaler 10, such as for use with a canister 11 of the pMDI type for dispersal of a medication or medicament to a patient. Various types of medications or medicaments can be utilized, such as various drug solutions, suspensions, aerosols, or the like. The medication inhaler includes a canister holder 12 that provides for compact storage and further allows a pMDI canister 11 to be left installed in the inhaler 10 during storage. In addition or alternatively, the medication inhaler 10 can be utilized with various other types of canisters.

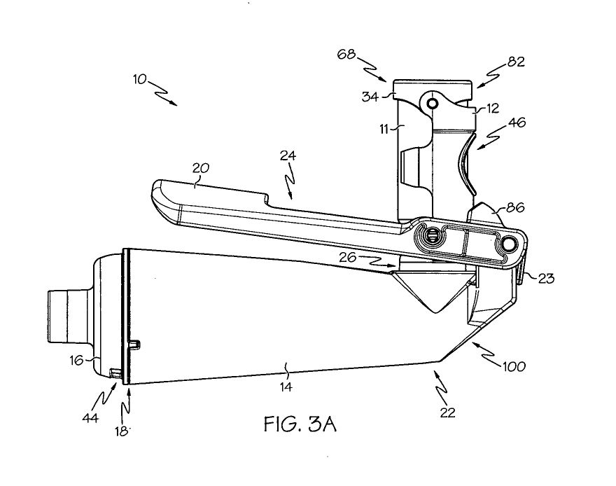

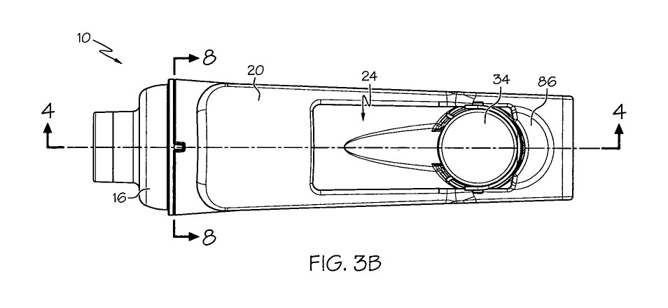

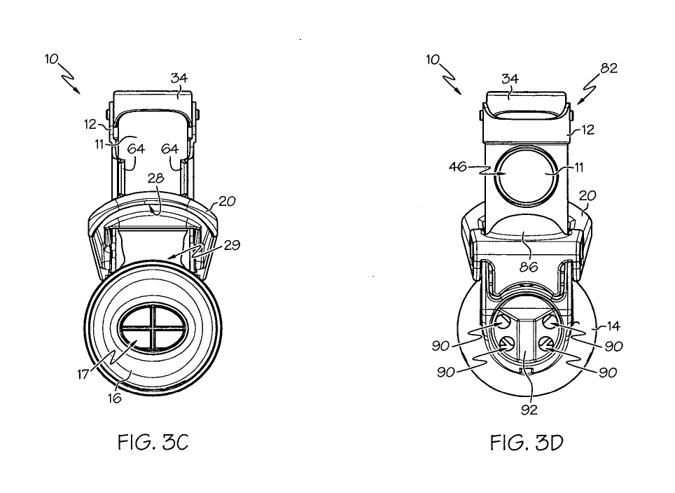

As shown in FIGS. 2 and 3A, the inhaler 10 also includes an elongated chamber body 14 having a proximal end 18 and a distal end 22. The chamber body 14 further includes a hollow spacer chamber 32 located within the chamber body 14 that extends between the proximal and distal ends 18, 22. In the illustrated example, the hollow spacer chamber 32 can taper from a relatively larger cross-sectional area adjacent the proximal end 18 to a relatively smaller cross-sectional area adjacent the distal end 22. Alternatively, the hollow spacer chamber 32 can include various other geometries. In one example, the hollow spacer chamber 32 can have a generally cylindrical geometry with a single cross-sectional area. It is to be appreciated that the hollow spacer chamber 32 can vary among various cross-sectional areas along the chamber body 14 with various degrees of variation (e.g., gradual, sharp, constant, etc.). In yet another example, the hollow spacer chamber 32 can include a frusto-conical geometry. As will be discussed herein, the hollow spacer chamber 32 can receive the medicine from a pMDI canister 11, and the geometry of the chamber 32 can facilitate mixture with an air supply.

Further, a mouthpiece 16 can be attached to the proximal end 18 of the inhaler 10. The mouthpiece 16 can be shaped to fit a mouth of a patient and can include a breathing port 17 to provide fluid communication between a user's mouth (not shown) and the hollow spacer chamber 32. As shown in FIG. 2, the mouthpiece 16 can be removably attached to the inhaler 10 in various manners to permit cleaning, maintenance, and/or replacement of the mouthpiece 16. As also shown, the mouthpiece 16 includes a mounting structure or bead 19A for engagement with a corresponding mounting structure or bead 19B provided on the proximal end 18 of the chamber body 14. In the illustrated example, the mounting structures 19A, 19B permit a snap-fit connection. Alternatively, other connection methods can also be used, such as an interference fit, fasteners, adhesives, or the like. The mounting structure 19A, 19B can also be configured to properly align the mouthpiece 16 with respect to the chamber body 14. Additionally, the mouthpiece 16 can also include an exhalation port 44, as will be discussed further herein.

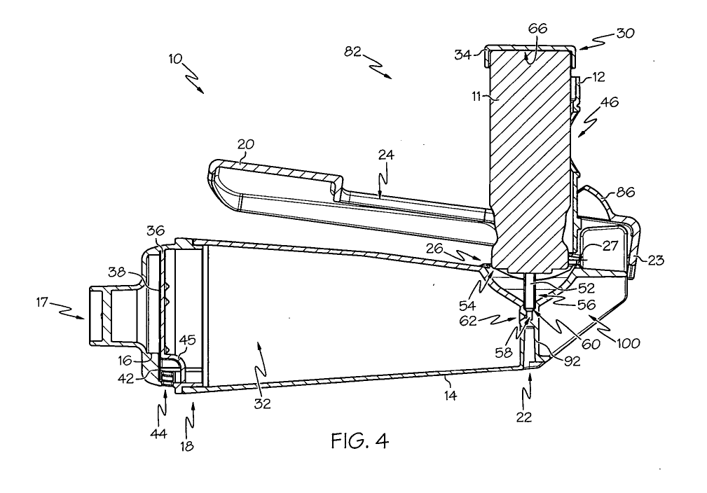

As shown in FIGS. 2 and 4, a one-way valve assembly 35 can be provided between the mouthpiece 16 and the hollow spacer chamber 32 within the chamber body 14. In the illustrated example, the valve assembly 35 includes a valve plate 36 and at least one resilient inhalation valve member 38 pivotally attached to the valve plate 36 to cover respective valve seats 40. As shown, a pair of valve members 38 can be pivotally attached to the valve plate 36, such as though one or more plugs received within holes of the valve plate 36. Various other attachment methods can also be used, such as adhesives, various fasteners, welding, and/or the valve members 38 can even be formed with the valve plate 36. When the patient inhales through the mouthpiece 16, the reduced pressure within the mouthpiece 16 causes the inhalation valve members 38 to move away from their valve seats 40, allowing air and vaporized medicament to flow from the chamber 32, through valve seats 40 and into the mouthpiece 16. However, upon exhalation by the patient, the one-way valve assembly 35 can inhibit exhaled air and/or medicament to flow back into the chamber 32. Additionally, by providing two valve members 38, as shown, each valve member 38 requires less force to open. Alternatively, one or any number of valve members 38 can be provided.

The valve assembly 35 can further include a resilient exhalation valve member 42 pivotally attached to the valve plate 36 to cover the exhalation port 44 of the mouthpiece 16. As with the pair of valve members 38, the valve plate 36 can include a separation member 45 configured to direct a patient's exhalation breath towards the exhalation port 44 of the mouthpiece. As before, the exhalation valve member 42 can be pivotally attached to the valve plate 36 in various manners, such as though one or more plugs received within holes of the valve plate 36, adhesives, various fasteners, welding, and/or can even be formed with the valve plate 36. Thus, during inhalation, the exhalation valve member 42 can be forced against a front wall of the mouthpiece 16, keeping the exhalation port 44 closed. When the patient exhales, an increase in pressure within the mouthpiece 16 can cause the inhalation valve members 38 to be forced against their respective valve seats 40, preventing the breath of the patient from entering the chamber 32. At the same time, the exhalation valve member 42 moves away from the wall of the mouthpiece 16, opening the exhalation port 44, and allowing the exhalation of the patient to be vented to the atmosphere.

The inhaler 10 can further include an actuating lever 20 that can be pivotally attached to a distal end 22 of the chamber body 14. The actuating lever 20 can include mounting structure 21A for engagement with corresponding mounting structure 21B of the distal end 22 of the chamber body 14, such as pins for engagement with holes. Additionally, the mounting structure 21A, 21B can provide a permanent connection, or even a removable connection to permit cleaning of various elements of the inhaler 10 (e.g., chamber body 14, actuating lever 20, etc.). Further, the actuation lever 20 can include geometry corresponding to that of the chamber body 14. For example, as shown in FIG. 3C, a portion of the exterior surface 29 of the chamber body 14 can include a generally convex geometry, and a portion 28 of the actuation lever 20 can include a generally concave geometry that is configured for a mating relationship with the convex geometry of the chamber body 14. As such, the actuation lever 20 can fold against the chamber body 14 to thereby reduce the overall size of the inhaler 10 during storage. Additionally, lever 20 can also include a stop 23 for engagement with a portion of the chamber body 14 to limit vertical movement of the lever 20.

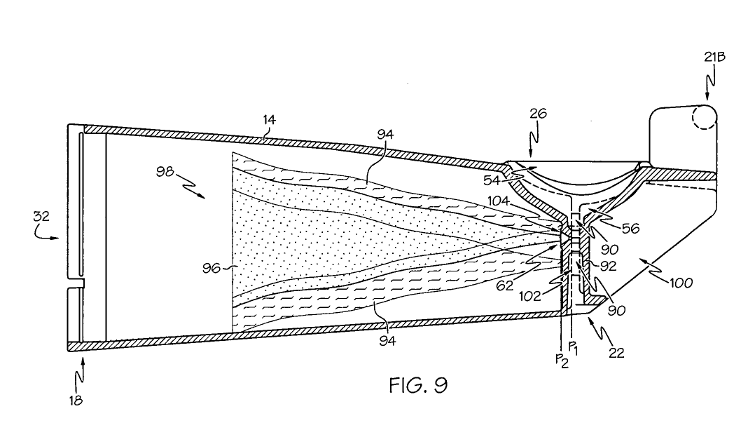

Turning now to FIG. 4, the chamber body 14 can further include an inlet portion 26 for receiving a valve stem 52 of the pMDI canister 11. The inlet portion 26 can be provided on the chamber body 14 near the distal end 22. As shown in FIGS. 4 and 9, the inlet portion 26 can include a first end 54 for receiving the valve stem 52 and a second end 56 for engagement with the valve stem 52. Additionally, the inlet portion 26 can taper from a relatively larger cross-sectional area adjacent the first end 54 to a relatively smaller cross-sectional area adjacent the second end 56. It is to be appreciated that the inlet portion 26 can vary among various cross-sectional areas between the first and second ends 54, 56 with various degrees of variation (e.g., gradual, sharp, constant, etc.). In yet another example, the inlet portion 26 can include a frusto-conical geometry. The geometry of the inlet portion 26 can permit self-alignment of the valve stem 52 relative to the chamber body 14 during usage.

When a pMDI canister is installed in the canister holder 12 and the canister holder 12 is fully extended (e.g., in the operational position 82), the tapered and/or frusto-conical geometry of the inlet portion 26 can guide the valve stem 52 from the first end 54 towards the second end 56 as the lever 20 is moved toward the chamber body 14. As the lever 20 is squeezed by a patient, an end stop 30 of the canister holder 12 can force the canister against the inlet portion 26, causing the valve stem 52 to be depressed, which releases medicament from the canister into the hollow spacer chamber 32 of the chamber body 14.

Additionally, as shown, the second end 56 of the inlet portion 26 can include a hole 58. The hole 58 can include a shoulder 60 for engagement with the valve stem 52. Additionally, the hole 58 can be in fluid communication with a nozzle 62 disposed within the hollow spacer chamber 32. The nozzle 62 can be configured to direct a plume of medication from the pMDI canister 11 into the hollow spacer chamber 32, and can include various geometries corresponding to desired performance characteristics of the inhaler 10. Thus, as the lever 20 is squeezed by a patient, the tapered geometry of the inlet portion 26 can be configured to drive the valve stem 52 into the hole 58. Once in the hole, engagement of valve stem 52 with the shoulder 60 can cause the medication to be dispersed though the nozzle 62 and into the hollow spacer chamber 32.

As stated previously, the inhaler 10 can further include a canister holder 12 configured to retain the pMDI canister 11 in an operational position relative to the chamber body 14. The canister retainer 12 can include various features for retention of a pMDI canister 11. In one example, the canister retainer 12 can include one or more retention arms 64 configured to grip the pMDI canister 11 upon insertion into the canister holder 12. For example, a pair of retention arms 64 can be provided in a spaced apart relationship, such that a pMDI canister can be inserted therebetween. The retention arms 64 can be spaced apart a distance less than the width of the pMDI canister 11 so as to inhibit inadvertent removal of the canister 11 from the inhaler 10. In another example, the canister retainer 12, including the arms 64, can include a resilient material, such as a resilient polymer, metal, or the like to wrap about and/or conform to the exterior of the pMDI canister 11 to provide greater retention. It is to be appreciated that, conventionally, various pMDI canisters are manufactured in standard sizes, such as standard outer diameters of the pMDI canisters 11 that are generally similar, though the resilient material of the canister holder 12 can permit retention of various size canisters 11.

As stated above, the canister retainer 12 can further include an end stop 30 that, in cooperation with the lever 20, can facilitate movement of the canister 11. In the shown examples, the end stop 30 is configured to act upon an end 66 of the canister 11. Generally, various pMDI canisters are manufactured in standard sizes. In many cases, the outer diameters of the pMDI canisters 11 are generally similar. However, it is common to have pMDI canisters with various lengths. Often, it is common to have various medications provided in either of a relatively shorter canister and a relatively longer canister, though various other sizes can also be used.

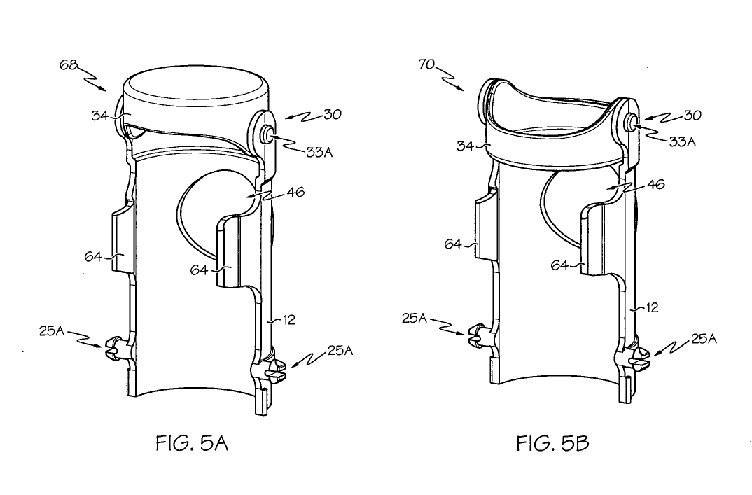

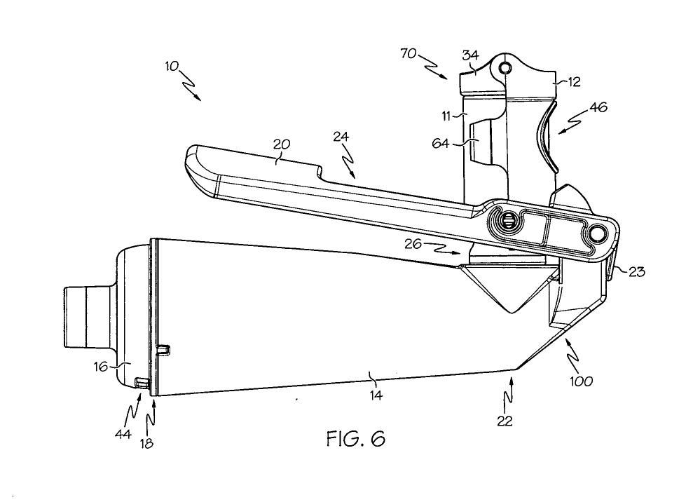

Thus, the canister retainer 12 can be further adapted to accommodate canisters 11 of various lengths. Though the following example illustrates accommodation of a first pMDI canister having a first size (e.g, a first length) and a second pMDI canister having a second size (e.g., a second length), the canister retainer 12 can be configured to accommodate various numbers of canisters 11 having various lengths. In one example, as shown in FIGS. 5A-5B, the canister retainer 12 can include a canister adapter 34 attached to the canister retainer 12 and adjustable from at least a first configuration 68 to a second configuration 70. The first configuration 68 can be associated with the relatively longer pMDI canister 11, while the second configuration 70 can be associated with the relatively shorter pMDI canister 11. Thus, either of the first or second configurations 68, 70 can be adapted to position the valve stem 52 of the associated canister 11 adjacent the inlet portion 26 of the chamber body 14. For example, FIG. 3A illustrates an inhaler 10 with the canister adapted 34 in the first configuration 68 for use with a relatively longer canister 11. Similarly, FIG. 6 illustrates an inhaler 10 with the canister adapted 34 in the second configuration 70 for use with a relatively longer canister 11.

The canister adapter 34 can be attached to the canister retainer 12 in various ways and adjusted between the first and second configurations 68, 70 (or various other configurations) in various manners. In one example, as shown in FIGS. 5A and 5B, the canister adapter 34 can be pivotally attached to the canister retainer 12. The canister adapter 34 can include mounting structure 33A for engagement with corresponding mounting structure 33B of canister retainer 12, such as pins for engagement with holes. Additionally, the mounting structure 33A, 33B can provide a permanent connection, or even a removable connection to permit cleaning of various elements of the inhaler 10 (e.g., canister retainer 12, chamber body 14, actuating lever 20, etc.). As illustrated by the two figures, the canister adapter 34 can be pivoted between the first and second configurations 68, 70. Thus, the canister adapter 34 can be quickly and easily adjusted for use with various canisters 11 of various lengths.

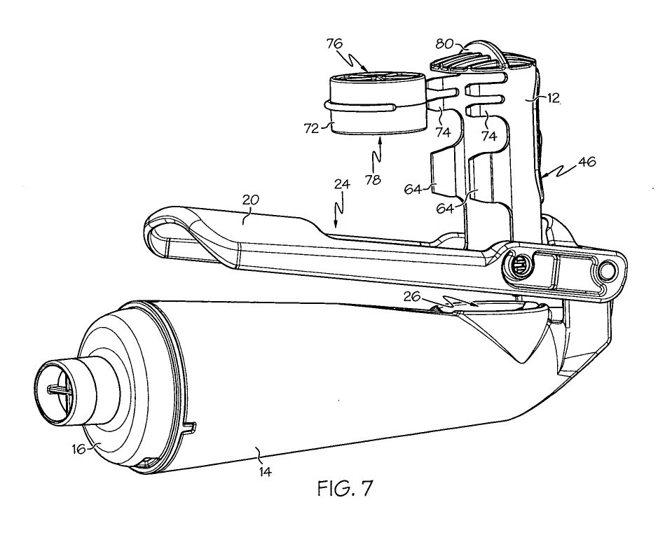

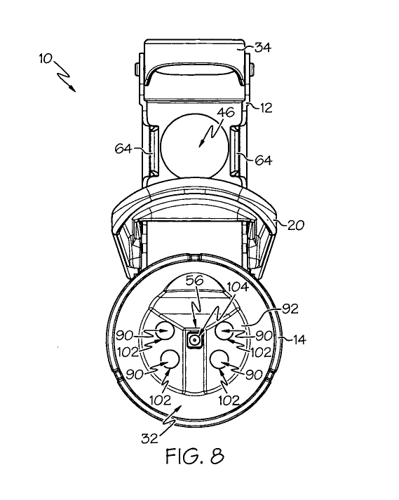

In another example, as shown in FIG. 7, an alternate canister adapter 72 can be removably attached to the canister holder 12 for adjustment from the first configuration 68 to the second configuration 70. For example, the canister retainer 12 can include additional retention arms 74 or the like for engagement with and retention of the alternate adapter 72. The alternate adapter 72 can be removably attached to the canister holder 12 in various manners, such as a snap-fit connection, interference fit, fasteners, adhesives, or the like. Either or both of the adapter 72 and retention arms 74 can further include alignment structure. Further, as shown, the adapter 72 can include a hollow interior having an end cap 76 on one side and an opening 78 on the opposite side so that when attached to the canister holder 12 in a predetermined position, a longer canister can be accommodated. In this way, the adapter 72 can be stored when not needed for a shorter canister. Alternatively, the adapter 72 can be rotated upside down such that the end cap 76 can accommodate a shorter canister. In addition or alternatively, the adapter 72 can be removed to accommodate a longer canister 11. For example, the canister retainer 12 can also include a removable or non-removable top 80 to further retain the canister 11, or even the adapter 72. It is to be appreciated that various other canister adapters can be attached to the canister retainer 12 in various other manners, and can even be fixedly attached to the canister retainer 12.

Additionally, a finger hole 46 can be provided in a sidewall of the canister holder 12. An empty canister 11 can easily be removed from the inhaler 10 by the patient by inserting a finger into the finger hole 46, and pushing the canister out of the canister holder 12. Other holes or the like (not shown) can also be provided in the canister holder 12 to assist the patient in removing the adapter 34.

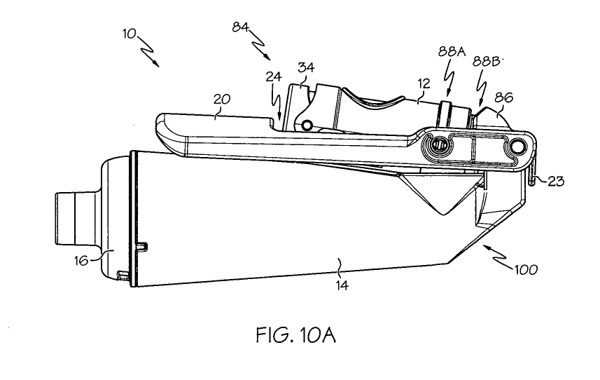

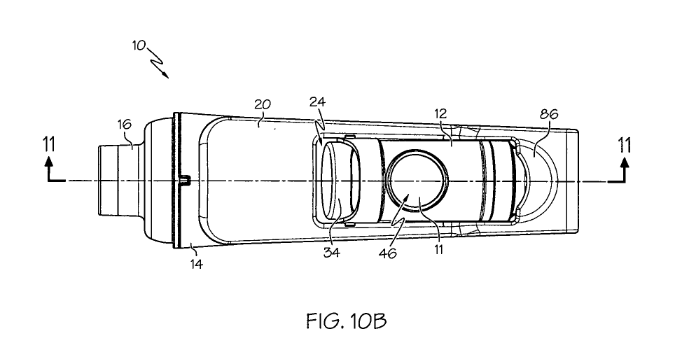

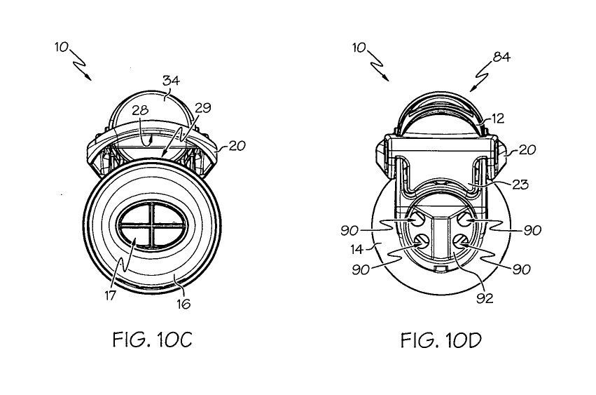

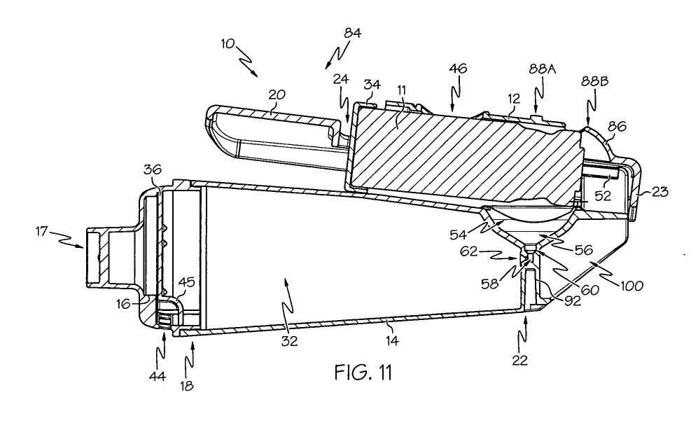

Further still, the canister holder 12 can be pivotally attached to the lever 20 for movement relative to the lever 20 between an operational position 82, as shown in FIGS. 3A-4, and a stored position 84, as shown in FIGS. 10A-11. As such, a user is not required to remove the canister 11 from the inhaler 10 until the canister 11 is in need of replacement (e.g., when it is empty), though are free to do so for various reasons. As shown, the canister holder 12 can include mounting structure 25A for engagement with corresponding mounting structure 25B of the lever 20, such as pins for engagement with holes. Additionally, the mounting structure 25A, 25B can provide a permanent connection, or even removable connection to permit cleaning of various elements of the inhaler 10 (e.g., chamber body 14, actuating lever 20, canister holder 12, etc.).

The lever 20 can also include an elongated opening 24 for receiving the canister retainer 12. The elongated opening 24 can include an aperture extending through the lever 20, though it can also include a depression or the like. Thus, the elongated opening 24 can allow the canister holder 12 to be folded down and stored at least partially within a space provided between the lever 20 and the chamber body 14. The operational position 82 can be a raised position such that the valve stem 52 of the canister 11 is adjacent the inlet portion 26 of the chamber body 14. The stored position 84 can be a lowered position such that at least a portion of the upper end 66 of the canister 11 is received within the elongated opening 24. To accomplish movement between the operational and stored positions 82, 84, in one example, a user can lift the lever 20 upwards, rotate the canister retainer 12 accordingly, and lower the lever 20 back downwards towards the chamber body 14.

The inhaler 10 can include additional structure to facilitate movement between the operational and stored positions 82, 84. In one example, as shown in FIG. 2, the inlet portion 26 can include a groove 27, depression, or the like to provide additional clearance for the valve stem 52 during movement between the operational 82 and stored 84 positions. In another example, the chamber body 14 can further include a protective cover 86 being configured to extend a distance over the valve stem 52 of the canister 11 when the canister retainer 12 is in the stored position 84. As such, the protective cover 86 can act as a protective shroud to inhibit inadvertent actuation of the valve stem 52, such as when the inhaler 10 is being carried in a pocket, purse, bag, or the like. The protective cover 86 can also inhibit contamination of the valve stem 52 from outside sources.

In another example, the inhaler 10 can include structure to maintain the canister retainer 12 in either of the operational and stored positions 82, 84. For example, the canister retainer 12 can include a first maintaining element 88A and the chamber body 14 can include a second maintaining element 88B. Engagement of the maintaining elements 88A, 88B can inhibit movement of the canister retainer 12 from the operational position 82 to the stored position 84. As shown in FIG. 11, the first maintaining element 88A can include a projection or the like, and the second maintaining element 88B can include a portion of the protective cover 86. For example, when moving the canister retainer 12 from the stored position 84 to the operational position 82, the first maintaining element 88A can be configured to relatively easily move past the second maintaining element 88B in a snap-lock arrangement or the like. However, when moving the canister retainer 12 from the operational position 82 to the stored position 82, the maintaining elements 88A, 88B can further be configured such that it is relatively difficult for the first maintaining element 88A to move past the second maintaining element 88B (e.g., removal from underneath the protective cover 86). As such, the maintaining elements 88A, 88B can facilitate a proper positioning of the canister 11 relative to the inlet portion 26 during operation. However, it is to be appreciated that the maintaining element 88A, 88B can also permit some movement of the canister retainer 12, such as approximately 10° from the vertical or the like. Additionally, it is to be appreciated that the inhaler 10 can further include additional maintaining elements (not shown) configured to maintain the canister retainer 12 in the stored position 82 (e.g., inhibit movement from the stored position 84 to the operational position 82). Various other maintaining elements 88A, 88B can also be used to facilitate selective maintenance of either the operational or stored positions 82, 84.

As stated previously, the amount of medicament reaching the lungs has conventionally been inconsistent, depending on many factors, including mixture of the medication with the stream of air entering the patient's lungs, and how much of the medicament is deposited on surfaces of the inhalation apparatus, the mouth and oropharyngeal area of the patient. Thus, the inhaler 10 can also include structure to facilitate delivery of the medication to a patient.

For example, the inhaler 10 can include one or more fresh air inlets 90 providing fluid communication between the atmosphere and the hollow spacer chamber 32. For example, the air inlets 90 can draw in fresh air from a rear inlet portion 100 of the chamber body 14. As shown in FIG. 8, the inhaler 10 can include four fresh air inlets 90 arranged about the nozzle 62, though various numbers of inlets 90 can be arranged in various manners. The fresh air inlets 90 can deliver a volume of fresh air from the atmosphere into the hollow spacer chamber 32, such as upon inhalation by the patient. Thus, when a patient inhales, the volume of fresh air provided by the fresh air inlets 90 can enhance delivery of the medication from the pMDI canister 11 to the patient. In addition or alternatively, the volume of fresh air provided through the fresh air inlets 90 can act to warm the air/medication mixture before delivery to the patient through the mouthpiece 16 to provide greater comfort to the patient.

The volume of fresh air provided by the fresh air inlets 90 can facilitate mixture of the medication with the fresh air to provide an enhanced medication delivery to the patient and reduce the amount of medication deposited on surfaces of the inhaler 10 and/or the mouth and oropharyngeal area of the patient. In one example, the inhaler 10 can include materials that resist accumulation of the medication on the interior surfaces. For example, the chamber body 14 and/or mouthpiece 16 can include various anti-static materials, such as a suitable polycarbonate material or the like, that resist a deposition of medication thereon. However, it is to be appreciated that the various other elements of the inhaler 10 that do not have direct contact with the medication may or may not include anti-static materials, and may be formed of other generally rigid materials, such as plastic, metal, hard rubber, or the like.

In another example, the volume of fresh air can facilitate mixture with the medication fluid such that the medication fluid is generally uniformly distributed within the volume of fresh air before the medication fluid passes from the hollow spacer chamber 32 and into the mouthpiece 16. Turning briefly to the example shown in FIG. 9, fresh air streams 94 provided by the fresh air inlets 90 can mix with the medication stream 96 provided by the nozzle 62. As shown, the medication fluid can be generally uniformly mixed with the volume of fresh air within a portion 98 of the hollow spacer chamber 32 before the medication passes into the mouthpiece 16 for inhalation by the patient. It should be appreciated that the shapes of the air stream 94 and the medication stream 96 may vary from the illustration, which is partially schematic and is shown for the purposes of illustration only.

It is to be appreciated that the geometry of the hollow spacer chamber 32 can further facilitate mixture of the air and medication. In one example, mixture can be enhance by the flow of the air and medication streams 94, 96 from the generally smaller cross-sectional area adjacent the distal end 22 of the hollow spacer chamber 32 to the generally larger cross-sectional area adjacent the proximal end 18. In another example, the frusto-conical shape of the hollow spacer chamber 32 can act as a diffuser to facilitate the mixture. The geometry of the hollow spacer chamber 32 can act to decrease the velocity of the air and medication streams 94, 96 towards the proximal end 18 (e.g., near the mixing portion 98) to enhance mixture of the air and medication. The geometry can also act to make the air flow relatively less laminar (e.g., as opposed to a cylinder having a generally constant inner diameter) through the hollow spacer chamber 14 to further facilitate mixture of the medication and air. However, because the hollow spacer chamber 32 can have various geometries, it is also to be appreciated that the mixture portion 98 of the hollow spacer chamber 32 can be disposed at various locations within the hollow spacer chamber 32.

Additional structure can further facilitate mixture of the air and medication. For example, each fresh air inlet 90 can include an outlet 102 generally located towards the distal end 22 of the chamber body 14. As shown in FIGS. 8 and 9, the fresh air inlets 90 can be formed through a portion of the chamber body 14, such as a plate 92. The plate 92 can extend across a portion of the hollow chamber body 32, and can even extend completely across so as to form a wall. Additionally, the plate 92 can cooperate with the inlet portion 26 and can include the hole 58, shoulder 60, and/or even the nozzle 62.

In one example, the fresh air inlets 90 can be formed as holes extending through the plate 92. Thus, the outlet 102 of each fresh air inlet 90 can be located on a face of the plate 92. The fresh air inlets 90 can also include various other structures and/or geometries, such as a tapered geometry (not shown). For example, a tapered geometry can provide the fresh air inlets 90 with a nozzle (not shown) to alter the flow pattern of the fresh air, such as by modifying the air stream velocity, plume shape, plume direction, plume orientation, etc. It is to be appreciated that such other structures and/or geometries can be included with the outlet 102, or even other portions of the fresh air inlets 90.

Additionally, the medication dispersal nozzle 62 can also include an outlet 104 located towards the distal end 22 of the chamber body and proximate the outlet 102 of the fresh air inlet. As shown in FIGS. 8 and 9, the outlets 102, 104 can be located relatively close to each other (e.g., each outlet 102 being located a relatively small horizontal and vertical distance from the nozzle outlet 104).

Further, the outlets 102, 104 can be offset from each other a relatively small distance along a longitudinal axis of the chamber body 14. For example, the outlet 102 of the fresh air inlets 90 can be located along a first plane P₁, while nozzle outlet 104 can extend along a second plane P₂, and the two planes P₁, P₂ can be spaced a distance from each other. In one example, as shown in FIG. 9, the two planes P₁, P₂ can be spaced a distance of approximately 1/16 of an inch, though various other offsets can also be used. It is to be appreciated that the fresh air outlets 102 can be spaced in front of or behind the nozzle outlets 104, though too great of an offset can cause reduced mixture of the air and medication.

In addition or alternatively, the first plane P₁ can be substantially coplanar with the second plane P₂. It is to be appreciated that the term “coplanar” includes, but is not limited to, being geometrically coplanar, and also includes two planes that are nearly geometrically coplanar, but are offset a relatively small distance (e.g., by design or as a result of a manufacturing process). Thus, the air outlets 102 can also be proximate the nozzle outlet 104 along the longitudinal axis of the chamber body 14. Accordingly, the proximate dispositions of the nozzles 102, 104 can permit the plumes of the fresh air streams 94 and the plume of the medication 96 to originate at similar locations within the hollow spacer chamber 32. The similar origination points can further facilitate mixture of the medication and the air stream 94 within the hollow spacer chamber 32 (e.g., near the mixture point 98 discussed previously).

An example operation of the inhaler 10 will now be discussed. In use, the patient can open the inhaler 10 by elevating the lever 20 and pivoting the canister holder 12 away from the lever 20 to the operational position 82. A canister 11 can be inserted into the canister holder 12 if not already installed, and/or the canister adapter 34 can be adjusted appropriately for use with a particular sized canister 11. The mouthpiece 16 can then be inserted into the patient's mouth. The patient can then squeeze the lever 20 toward the chamber body 14, causing the valve stem 52 to engage the second end 56 of the inlet portion 26 (e.g., engage the shoulder 60) to thereby activate the canister and deliver a dose of medicament into the hollow spacer chamber 32 through the nozzle 62. Prior to, simultaneous with, or subsequent to the activation of the pMDI canister 11, the patient can inhale through the mouthpiece 16, causing the inhalation valve members 38 to open, whereby the medicament is delivered to the patient. It is to be appreciated that it can be beneficial for the patient to inhale prior to activation of the canister 11 to provide a fresh air stream for increased mixture with the medication as discussed herein. Additionally, the inhaler 10 can be effectively operated with one-handed operation, and such one-handed operation can contribute to increased coordination of the timing of the medication release and inhalation by the patient to improve the effectiveness of the medication.

If the patient does not inhale the entire dose of medicament before stopping inhalation, the inhalation valve members 38 of the one-way valve assembly 35 can close and trap any remaining medicament in the hollow spacer chamber 32. The patient may then exhale, with or without withdrawing the mouthpiece 16. If the patient does not withdraw the mouthpiece, the exhalation valve member 42 can open to discharging the patient's breath through the exhalation port 44. The patient may then inhale through the mouthpiece 16 again, causing the remaining medicament to be delivered. This process can be repeated as many times as needed to deliver the entire dose of medicament from the chamber 32 (e.g., a patient can take one large inhalation or a plurality of smaller inhalations to receive the medication). When the patient is finished using the inhaler 10, the canister holder 12 can be pivoted to the stored position 84, thereby reducing the overall size of the inhaler 10 for storage.

The invention has been described with reference to various example embodiments. Obviously, modifications and alterations will occur to others upon a reading and understanding of this specification. It is intended to include all such modifications and alterations insofar as they come within the scope of the appended claims or the equivalents thereof.

What is claimed is:

1. A medication inhaler for delivery of a medication from a canister to a patient, the canister having a valve stem, the medication inhaler including:
a chamber body having a proximal end, a distal end, a fresh air inlet, a hollow spacer chamber located within the chamber body and tapering from a relatively larger cross-sectional area adjacent the proximal end to a relatively smaller cross-sectional area adjacent the distal end, and an inlet portion for receiving the valve stem of the canister;
a canister retainer configured to retain the canister in an operational position relative to the chamber body;
an actuation lever pivotally attached to the chamber body, the actuation lever being configured to actuate the valve stem of the canister to deliver a medication fluid into the hollow spacer chamber;
a mouthpiece for engaging a mouth of a patient, the mouthpiece being connected to the proximal end of the chamber body; and
a one-way valve disposed between the mouthpiece and the hollow spacer chamber and configured to provide one-way fluid communication between the hollow spacer chamber and the mouthpiece,
the fresh air inlet providing fluid communication between the atmosphere and the hollow spacer chamber to deliver a volume of fresh air from the atmosphere into the hollow spacer chamber upon inhalation by the patient for mixture with the medication fluid such that the medication fluid is generally uniformly distributed within the volume of fresh air before the medication fluid passes from the hollow spacer chamber and into the mouthpiece.

2. The medication inhaler of claim 1, wherein the hollow spacer chamber includes a generally frusto-conical geometry.

3. The medication inhaler of claim 1, wherein the fresh air inlet further includes an outlet being generally located towards the distal end of the chamber body, and the chamber body further includes a nozzle in fluid communication with the inlet portion and the hollow spacer chamber, the nozzle having an outlet being generally located towards the distal end of the chamber body and proximate the outlet of the fresh air inlet.

4. The medication inhaler of claim 3, wherein the outlet of the fresh air inlet is located along a first plane and the outlet of the nozzle is located along a second plane, the first plane being substantially coplanar with the second plane.

5. The medication inhaler of claim 1, further including a one-way valve disposed between the mouthpiece and the atmosphere and configured to provide one-way fluid communication between the mouthpiece and the atmosphere upon exhalation by the patient.

6. The medication inhaler of claim 1, wherein an exterior surface of the chamber body includes a convex curved geometry and a portion of the actuation lever includes a concave curved geometry, the concave curved geometry generally being configured for a mating relationship with the convex curved geometry.

7. The medication inhaler of claim 1, wherein the medication inhaler is configured to deliver a medication to a patient from at least one of a first canister having a first size and a second canister having a second size, the medication inhaler further including a canister adapter attached to the canister retainer and adjustable from a first configuration to a second configuration, the first configuration being adapted to position the valve stem of the first canister adjacent the inlet portion, and the second configuration being adapted position the valve stem of the second canister adjacent the inlet portion.

8. The medication inhaler of claim 1, wherein the inlet portion includes a first end for receiving the valve stem of the canister and a second end for engagement with the valve stem, the inlet portion tapering from a relatively larger cross-sectional area adjacent the first end to a relatively smaller cross-sectional area adjacent the second end.

9. The medication inhaler of claim 8, wherein the second end of the inlet portion includes a hole, the tapered geometry of the inlet portion being configured to drive the valve stem into the hole.

10. The medication inhaler of claim 1, wherein the actuation lever includes an elongated opening, and the canister retainer is pivotally attached to the actuation lever and is movable between a stored position and an operational position, the stored position being a lowered position such that at least a portion of an upper end of the canister is received within the elongated opening of the actuation lever, and the operational position being a raised position such that the valve stem of the canister is adjacent the inlet portion of the chamber body.

11. The medication inhaler of claim 1, wherein the chamber body includes an anti-static material.

12. A medication inhaler for delivery of a medication from a canister to a patient, the canister having a valve stem, the medication inhaler including:
a chamber body having a proximal end, a distal end, a hollow spacer chamber located within the chamber body, an inlet portion for receiving the valve stem of the canister, a nozzle in fluid communication with the inlet portion and the hollow spacer chamber, the nozzle having an outlet, and a fresh air inlet in fluid communication with the atmosphere and the hollow spacer chamber, the fresh air inlet having an outlet proximate the outlet of the nozzle;
a canister retainer configured to retain the canister in an operational position relative to the chamber body;
an actuation lever pivotally attached to the chamber body, the actuation lever being configured to actuate the valve stem of the canister to deliver a medication fluid into the hollow spacer chamber;
a mouthpiece for engaging a mouth of a patient, the mouthpiece being connected to the proximal end of the chamber body; and
a one-way valve disposed between the mouthpiece and the hollow spacer chamber and configured to provide one-way fluid communication between the hollow spacer chamber and the mouthpiece.

13. The medication inhaler of claim 12, wherein the outlet of the fresh air inlet is located along a first plane and the outlet of the nozzle is located along a second plane, the first plane being substantially coplanar with the second plane.

14. The medication inhaler of claim 12, wherein the hollow spacer chamber tapers from a relatively larger cross-sectional area adjacent the proximal end to a relatively smaller cross-sectional area adjacent the distal end.

15. The medication inhaler of claim 12, wherein the chamber body includes an anti-static material.

16. A medication inhaler for delivery of a medication to a patient from at least one of a first canister having a first size and a second canister having a second size, each of the first and second canisters having an upper end, a lower end, and a valve stem disposed at the lower end, the medication inhaler including:
a chamber body having a proximal end, a distal end, a hollow spacer chamber located within the chamber body, and an inlet portion for receiving the valve stem of one of the canisters;
a canister retainer configured to retain either of the canisters in an operational position relative to the chamber body;
an actuation lever pivotally attached to the chamber body, the actuation lever being configured to actuate the valve stem of the canister retained by the canister retainer to deliver a volume of medication fluid into the hollow spacer chamber;
a mouthpiece for engaging a mouth of a patient, the mouthpiece being connected to the proximal end of the chamber body;
a one-way valve disposed between the mouthpiece and the hollow spacer chamber and configured to provide one-way fluid communication between the hollow spacer chamber and the mouthpiece; and
a canister adapter attached to the canister retainer and adapted to engage the upper end of a canister, the canister adapter being adjustable from a first configuration to a second configuration, the first configuration being adapted to position the valve stem of the first canister adjacent the inlet portion, and the second configuration being adapted position the valve stem of the second canister adjacent the inlet portion.

17. The medication inhaler of claim 16, wherein the canister adapter is pivotally attached to the canister retainer for adjustment from the first configuration to the second configuration.

18. The medication inhaler of claim 16, wherein the canister adapter is removably attached to the canister retainer for adjustment from the first configuration to the second configuration.

19. The medication inhaler of claim 16, further including a nozzle in fluid communication with the inlet portion and the hollow spacer chamber, the nozzle having an outlet located along a first plane, and a fresh air inlet in fluid communication with the atmosphere and the hollow spacer chamber, the fresh air inlet having an outlet located along a second plane, the first plane being substantially coplanar with the second plane.

20. The medication inhaler of claim 16, wherein the inlet portion includes a first end for receiving the valve stem of the canister and a second end for engagement with the valve stem, the inlet portion tapering from a relatively larger cross-sectional area adjacent the first end to a relatively smaller cross-sectional area adjacent the second end.

21. The medication inhaler of claim 16, wherein the actuation lever includes an elongated opening, and the canister retainer is pivotally attached to the actuation lever and is movable between a stored position and an operational position, the stored position being a lowered position such that at least a portion of the upper end of the canister is received within the elongated opening of the actuation lever, and the operational position being a raised position such that the valve stem of the canister is adjacent the inlet portion of the chamber body.

22. A medication inhaler for delivery of a medication from a canister to a patient, the canister having a valve stem, the medication inhaler including:
a chamber body having a proximal end, a distal end, a hollow spacer chamber located within the chamber body, and an inlet portion having a first end for receiving the valve stem of the canister and a second end for engagement with the valve stem, the inlet portion tapering from a relatively larger cross-sectional area adjacent the first end to a relatively smaller cross-sectional area adjacent the second end;
a mouthpiece for engaging a mouth of a patient, the mouthpiece being connected to the proximal end of the chamber body;
a one-way valve disposed between the mouthpiece and the hollow spacer chamber and configured to provide one-way fluid communication between the hollow spacer chamber and the mouthpiece;
a canister retainer configured to retain the canister in an operational position relative to the chamber body; and
an actuation lever pivotally attached to the chamber body and being configured to move the valve stem of the canister into contact with the second end of the inlet portion to deliver a medication fluid into the hollow spacer chamber.

23. The medication inhaler of claim 22, wherein movement of the actuation lever towards the chamber body causes movement of the valve stem towards the second end of the inlet portion.

24. The medication inhaler of claim 23, wherein the second end of the inlet portion includes a hole, the tapered geometry of the inlet portion being configured to drive the valve stem into the hole.

25. The medication inhaler of claim 24, wherein the hole further includes a shoulder and is in fluid communication with a nozzle disposed within the hollow spacer chamber, engagement of the valve stem with the shoulder causing medication from the canister to be dispersed through the nozzle and into the hollow spacer chamber.

26. The medication inhaler of claim 22, further including a nozzle in fluid communication with the inlet portion and the hollow spacer chamber, the nozzle having an outlet, and a fresh air inlet in fluid communication with the atmosphere and the hollow spacer chamber, the fresh air inlet having an outlet proximal the outlet of the nozzle.

27. The medication inhaler of claim 22, wherein the medication inhaler is configured to deliver a medication to a patient from at least one of a first canister having a first size and a second canister having a second size, the medication inhaler further including a canister adapter attached to the canister retainer and adjustable from a first configuration to a second configuration, the first configuration being adapted to position the valve stem of the first canister adjacent the inlet portion, and the second configuration being adapted position the valve stem of the second canister adjacent the inlet portion.

28. The medication inhaler of claim 22, wherein the actuation lever includes an elongated opening, and the canister retainer is pivotally attached to the actuation lever and is movable between a stored position and an operational position, the stored position being a lowered position such that at least a portion of an upper end of the canister is received within the elongated opening of the actuation lever, and the operational position being a raised position such that the valve stem of the canister is adjacent the inlet portion of the chamber body.

29. A medication inhaler for delivery of a medication from a canister to a patient, the canister having a valve stem, the medication inhaler including:
a chamber body having a proximal end, a distal end, a hollow spacer chamber located within the chamber body, and an inlet portion having a first end for receiving the valve stem of the canister and a second end for engagement with the valve stem;
a mouthpiece for engaging a mouth of a patient, the mouthpiece being connected to the proximal end of the chamber body;
a one-way valve disposed between the mouthpiece and the hollow spacer chamber and configured to provide one-way fluid communication between the hollow spacer chamber and the mouthpiece;
a canister retainer configured to retain the canister in an operational position relative to the chamber body; and
an actuation lever pivotally attached to the chamber body and being configured to move the valve stem into contact with the inlet portion, a geometry of the inlet portion being configured to guide the valve stem towards the second end thereof, engagement of the valve stem with the second end causing actuation of the valve stem to deliver a medication fluid into the hollow spacer chamber.

30. The medication inhaler of claim 29, wherein second end of the inlet portion includes a hole in fluid communication with a nozzle disposed within the hollow spacer chamber, the hole having a shoulder, engagement of the valve stem with the shoulder causing medication from the canister to be dispersed through the nozzle and into the hollow spacer chamber.

31. The medication inhaler of claim 29, wherein the hollow spacer chamber tapers from a relatively larger cross-sectional area adjacent the proximal end to a relatively smaller cross-sectional area adjacent the distal end.

32. The medication inhaler of claim 29, wherein the chamber body further includes a nozzle in fluid communication with the inlet portion and the hollow spacer chamber, the nozzle having an outlet located along a first plane, and a fresh air inlet in fluid communication with the atmosphere and the hollow spacer chamber, the fresh air inlet having an outlet located along a second plane, the first plane being substantially coplanar with the second plane.

33. The medication inhaler of claim 29, wherein the medication inhaler is configured to deliver a medication to a patient from at least one of a first canister having a first size and a second canister having a second size, the medication inhaler further including a canister adapter attached to the canister retainer and adjustable from a first configuration to a second configuration, the first configuration being adapted to position the valve stem of the first canister adjacent the inlet portion, and the second configuration being adapted position the valve stem of the second canister adjacent the inlet portion.

34. The medication inhaler of claim 29, wherein the actuation lever includes an elongated opening, and the canister retainer is pivotally attached to the actuation lever and is movable between a stored position and an operational position, the stored position being a lowered position such that at least a portion of an upper end of the canister is received within the elongated opening of the actuation lever, and the operational position being a raised position such that the valve stem of the canister is adjacent the inlet portion of the chamber body.

35. A medication inhaler for delivery of a medication from a canister to a patient, the canister having an upper end, a lower end, and a valve stem disposed at the lower end, the medication inhaler including:
a chamber body having a proximal end, a distal end, a hollow spacer chamber located within the chamber body, and an inlet portion for receiving the valve stem of the canister;
a mouthpiece for engaging a mouth of a patient, the mouthpiece being connected to the proximal end of the chamber body;
a one-way valve disposed between the mouthpiece and the hollow spacer chamber and configured to provide one-way fluid communication between the hollow spacer chamber and the mouthpiece;
an actuation lever having an elongated opening and being pivotally attached to the chamber body, the actuation lever being configured to actuate the valve stem of the canister retained by the canister retainer to deliver a volume of medication fluid into the hollow spacer chamber; and
a canister retainer configured to retain the canister, the canister retainer being pivotally attached to the actuation lever and being movable between a stored position and an operational position, the stored position being a lowered position such that at least a portion of the upper end of the canister is received within the elongated opening of the actuation lever, and the operational position being a raised position such that the valve stem of the canister is adjacent the inlet portion of the chamber body.

36. The medication inhaler of claim 35, wherein the chamber body further includes a protective cover being configured to extend a distance over the valve stem of the canister when the canister retainer is in the stored position to inhibit inadvertent actuation of the valve stem.

37. The medication inhaler of claim 35, wherein the canister retainer includes a first maintaining element and the chamber body includes a second maintaining element, engagement of the first maintaining element with the second maintaining element inhibiting movement of the canister retainer from the operational position to the stored position.

38. The medication inhaler of claim 35, wherein the chamber body further includes a nozzle in fluid communication with the inlet portion and the hollow spacer chamber, the nozzle having an outlet located along a first plane, and a fresh air inlet in fluid communication with the atmosphere and the hollow spacer chamber, the fresh air inlet having an outlet located along a second plane, the first plane being substantially coplanar with the second plane.
